(19) United States
(12) Patent Application Publication
(10) Pub. No.: US 2002/0013546 A1
(43) Pub. Date: Jan. 31, 2002

(54) Method and device to improve aqueous humor drainage in an eye

(75) Inventors: Hans R. Grieshaber, Schaffhausen (CH); Robert Stegmann, Pretoria (ZA)

Correspondence Address: HENRY M FEIEREISEN, 350 FIFTH AVENUE, SUITE 3220, NEW YORK, NY, 10118

(73) Assignee: Grieshaber & Co. AG Schaffhausen

(21) Appl. No.: 09/954,415

(22) Filed: Sep. 17, 2001

Related U.S. Application Data
(63) Continuation of application No. 09/134,366, filed on Aug. 14, 1998.

(30) Foreign Application Priority Data
Aug. 15, 1997 (CH) 1997 1923/97
Mar. 10, 1998 (CH) 1988 0574/98

(51) Int. Cl. A61M 5/00; A61M 1/00

(52) U.S. Cl. 604/28; 604/540

(57) ABSTRACT

Method for improving aqueous humor drainage in an eye with a Schlemm's canal in which eye the aqueous humor secreted by the ciliary body is drained through the subsequent outflow pathways and to a device to maintain aqueous humor drainage. A medium injected in the form of a hydrophilic liquid or a biocompatible gaseous medium or a mixture of the hydrophilic liquid and the gaseous medium into Schlemm's canal, which is microsurgically exposed at one or more locations, locally expands Schlemm's canal by the increased pressure. With a support element subsequently implanted in the lumen of Schlemm's canal, the inner walls of the canal are supported and permanently held in an expanded position, whereby unimpeded drainage of the aqueous humor from Schlemm's canal through the subsequent outflow pathways is ensured.

### CROSS-REFERENCE TO RELATED APPLICATION

The present application claims priority under 35 U.S.C. 119 of Swiss Patent Application No. 1997 1923/97, filed Aug. 15, 1997, and Swiss Patent Application No. 1998 0574/98, filed Mar. 10, 1998, the disclosures of which are expressly incorporated by reference herein in their entireties.

### BACKGROUND OF THE INVENTION

1. Field of the invention

The present invention relates to a method and to a device to improve aqueous humor drainage in an eye, by which the aqueous humor secreted by the ciliary body is drained in the region of the iridocomeal angle through the trabecular mesh-work into the Schlemm's canal and from there through the subsequent natural outflow pathways.

2. Discussion of Background Information

For treatment of changes in the trabecular meshwork in front of Schlemm's canal which changes completely or only partially obstruct the drainage of the aqueous humor, method and apparatus are known, such as disclosed in U.S. Pat. Nos. 5,360,399 and 5,486,165, each of which is incorporated by reference in its entirety. Thus, as disclosed therein, a medium in the form of a highly viscous aqueous solution, preferably based on hyaluronic acid, is injected by an injection apparatus into Schlemm's canal through a probe introduced into Schlemm's canal, so that the trabecular meshwork is hydraulically expanded and its pores are opened. The openings thus created are coated by the highly viscous material as a protection against immediate reclosure.

Although in the aforementioned U.S. patents, the injection of a suitable medium into Schlemm's canal is disclosed as a technique for its opening, with these methods there is still the uncertainty that ultimately Schlemm's canal will close again as a result of various pathological changes. The drainage of the aqueous humor through Schlemm's canal and through the subsequent outflow pathways is thus significantly restricted or completely obstructed as a result of the deformation of Schlemm's canal.

### SUMMARY OF THE INVENTION

The object of the present invention is to provide a method and device to achieve improved, pressure-regulating circulation of the aqueous humor and its drainage from the eye, which is permanently maintained.

With regard to the method, the present invention is characterized in that Schlemm's canal, microsurgically exposed at one or more locations, is expanded in a first phase by a locally increased pressure, such as hydraulic pressure, and then, in a second phase, for example, is supported by appropriate elements implanted in the expanded lumen of Schlemm's canal, and thus permanently held in an expanded position.

The present invention also provides a device including an axially oriented support element supporting the inner wall of Schlemm's canal in the region of the locally expanded lumen and is placed such that the aqueous humor can permanently drain from Schlemm's canal through the subsequent natural outflow pathways of the eye.

Thus, the present invention is directed to a method for improving drainage of aqueous humor in an eye by which the aqueous humor secreted by the ciliary body is drained in the region of the iridocomeal angle through the trabecular meshwork in Schlemm's canal and from there through the subsequent natural outflow pathways, comprising expanding by localized increased pressure utilizing at least one of a gaseous and a liquid medium a lumen of Schlemm's canal which is microsurgically exposed at at least one location to obtain an expanded lumen; and holding the expanded lumen of Schlemm's canal in a permanently expanded position.

The holding the expanded lumen of Schlemm's canal in a permanently expanded position can comprise implanting at least one support element in the expanded lumen of Schlemm's canal. The implanting of the at least one support element in the expanded lumen of Schlemm's canal can comprise implanting at least one axially oriented support element permanently supporting the inside wall of Schlemm's canal.

The expanding can comprise injecting hydrophilic liquid medium into the lumen, injecting gaseous, biocompatible medium into the lumen, or a mixture of hydrophilic liquid medium and gaseous medium into the lumen.

The at least one support element can comprise a distal end, and a proximal end including a stop member, and the support element can be inserted in Schlemm's canal with the distal end being inserted into the expanded lumen, and the support element being tightly held against the inside wall of an incision by the stop member on the proximal end.

The at least one support element can comprise at least an external diameter that is plastically deformable, and the support element is capable of returning to an original shape due to thermal shape memory.

The at least one support element can be plastically deformable under normal human body temperature and after implantation is capable of returning to an original shape as a result of body temperature and shape memory.

The expanding can comprise expanding Schlemm's canal in at least two portions positioned at intervals axially spaced from each other, and the implanting at least one support element can comprise implanting a support element into each of the at least two portions.

The support element can comprise outflow openings distributed on the support element and connected with an interior of the support element, and the support element can be implanted so that the outflow openings are connected with natural outflow pathways of Schlemm's canal.

The implanting can include implanting a support element on each of two sides of the microsurgically exposed and expanded portion of Schlemm's canal.

There is also provided a device for holding an expanded lumen of Schlemm's canal in a permanently expanded position after Schlemm's canal is exposed in at least one place and expanded by a medium injected by an injection apparatus, comprising a support element comprising an elongated member that is axially and permanently orientable in Schlemm's canal, with the elongated member being constructed and arranged to permit aqueous humor to permanently drain from Schlemm's canal through outflow pathways of the eye.

The elongated member can comprise a long tube having a proximal end and a distal end, and the proximal end can include a contact collar flaring conically outward.

The elongated member can have an arcuate shape similar to that of Schlemm's canal. Moreover, the elongated member can be automatically deformable into an arcuate shape similar to that of Schlemm's canal. The elongated member can also longitudinally taper from the proximal end to the distal end.

The elongated member can comprise an interior and a plurality of outflow openings circumferentially and longitudinally distributed at intervals and connecting with the interior.

The elongated member can comprise two axially spaced torus-shaped end portions, each of the end portions including an outflow opening, and at least two webs circumferentially disposed at intervals between the end portions, and the at least two webs can comprise three webs.

The elongated member can comprise interlinked threads constructed and arranged as a hollow cylindrical network including outflow openings. The network can be plastically deformable, and returnable to an original shape by heating. The threads can be composed of various materials, such as, nickel-titanium alloy with thermal shape memory, or plastic having thermal shape memory. The threads can be individually wound with respect to each other forming the hollow cylindrical network. Moreover, the threads can be bound to each other in a helicoidal network. The hollow cylindrical network can be capable of automatically expanding. The elongated member can comprise interlinked metal wires wound together as a hollow cylindrical network including outflow openings. The elongated member can be composed of a coil spring of a single wire wound in a helix.

The elongated member can comprise a hollow cylindrical element, which can be composed of a biocompatible material, such as a biocompatible material selected from the group consisting of plastic, stainless steel, noble metal, such as silver, gold and platinum, and biological material.

The hollow cylindrical element can be coated with material capable of generating a desired biological reaction, or coated with material capable of preventing an adverse biological reaction.

The hollow cylindrical element can flexible for automatic adaptation to the lumen of Schlemm's canal in a direction of a theoretical longitudinal axis.

The hollow cylindrical element can be connectable to the injection apparatus via a separable connection.

The support element can comprise a separable probe of the injection apparatus.

The support element can comprise a hollow cylindrical support element capable of being pushed onto a probe of the injection apparatus, and can be implanted by axial movement of the probe relative to the support element for stable positioning into the expanded portion of Schlenun's canal.

### BRIEF DESCRIPTION OF THE DRAWINGS

The present invention is further described in the detailed description which follows, in reference to the noted plurality of drawings by way of non-limiting examples of preferred embodiments of the present invention, in which like reference numerals represent similar parts throughout the several views of the drawings, and wherein:

FIG. 1 shows a cross-section of the anterior section of the eye depicted schematically in a vertical plane;

FIG. 2 shows a portion of the eye depicted schematically with a scleral lamella dissected and folded upward and an exposed portion of Schlemm's canal;

FIG. 3 shows an enlarged, schematically depicted portion of the eye according to FIG. 2 with an injection probe inserted into the partially exposed Schlemm's canal to expand the canal;

FIG. 4 shows the portion of the eye according to FIG. 3 with a first embodiment of a support element implanted in one portion of Schlemm's canal in the form of a cylindrical prosthesis;

FIG. 5 shows the first embodiment of a support element as illustrated in FIG. 4, depicted in a perspective view and enlarged;

FIG. 6 shows a front view of the support element according to FIG. 5 in partial cross-section;

FIG. 7 shows the portion of the eye according to FIG. 3 with a support element implanted in the other portion of Schlemm's canal and designed as a second exemplary embodiment;

FIG. 8 shows the support element according to FIG. 7 depicted as the second exemplary embodiment, enlarged and in partial cross-section;

FIG. 9 shows a third embodiment of the support element to be implanted in Schlemm's canal;

FIG. 10 shows the support element according to FIG. 9 depicted in a side view along the line X-X and in cross-section;

FIG. 11 shows a fourth embodiment of the support element to be implanted in Schlemm's canal; and

FIG. 12 shows a further embodiment of the support element to be implanted in Schlemm's canal.

### DETAILED DESCRIPTION OF THE INVENTION

The particulars shown herein are by way of example and for purposes of illustrative discussion of the preferred embodiments of the present invention only and are presented in the cause of providing what is believed to be the most useful and readily understood description of the principles and conceptual aspects of the invention. In this regard, no attempt is made to show structural details of the invention in more detail than is necessary for the fundamental understanding of the invention, the description taken with the drawings making apparent to those skilled in the art how the several forms of the invention may be embodied in practice.

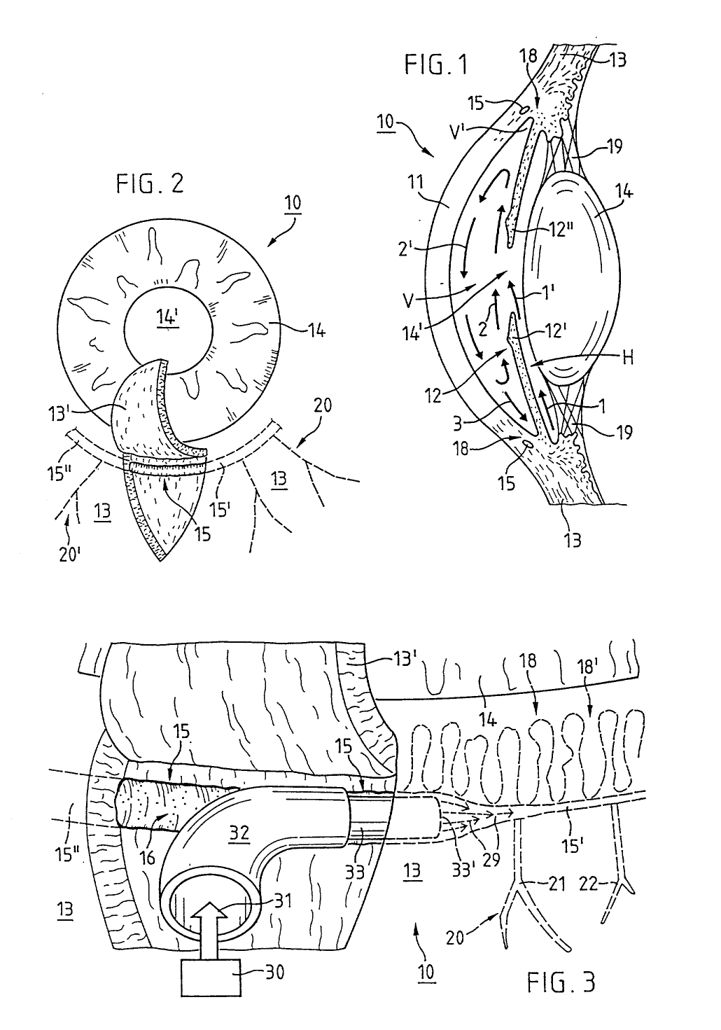

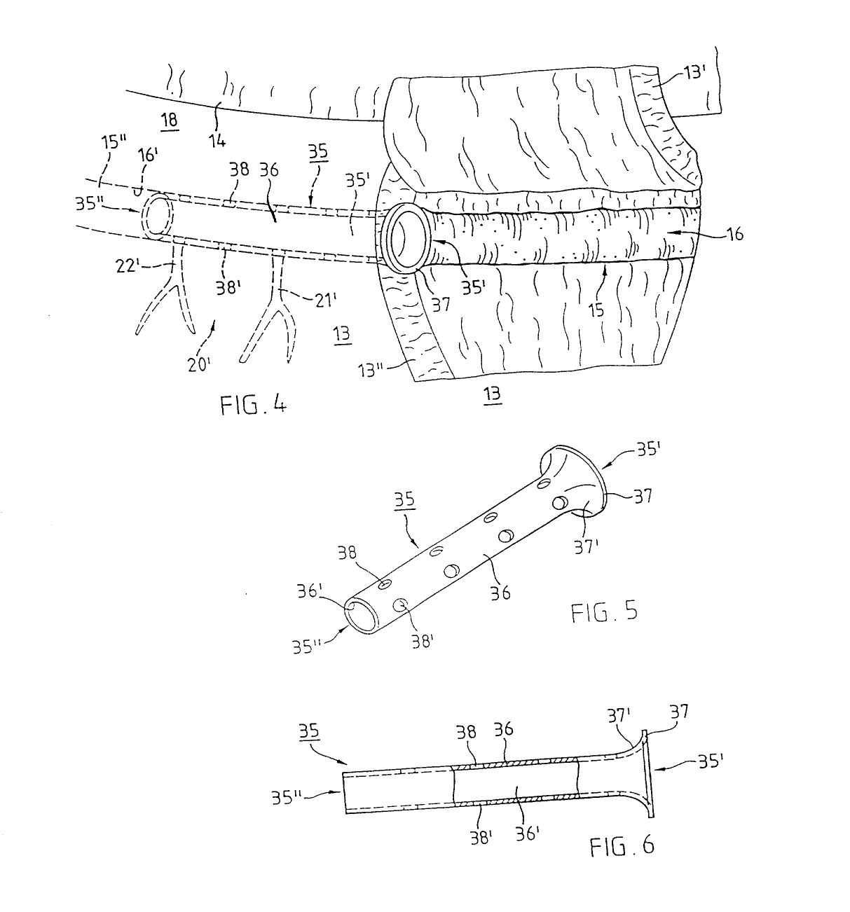

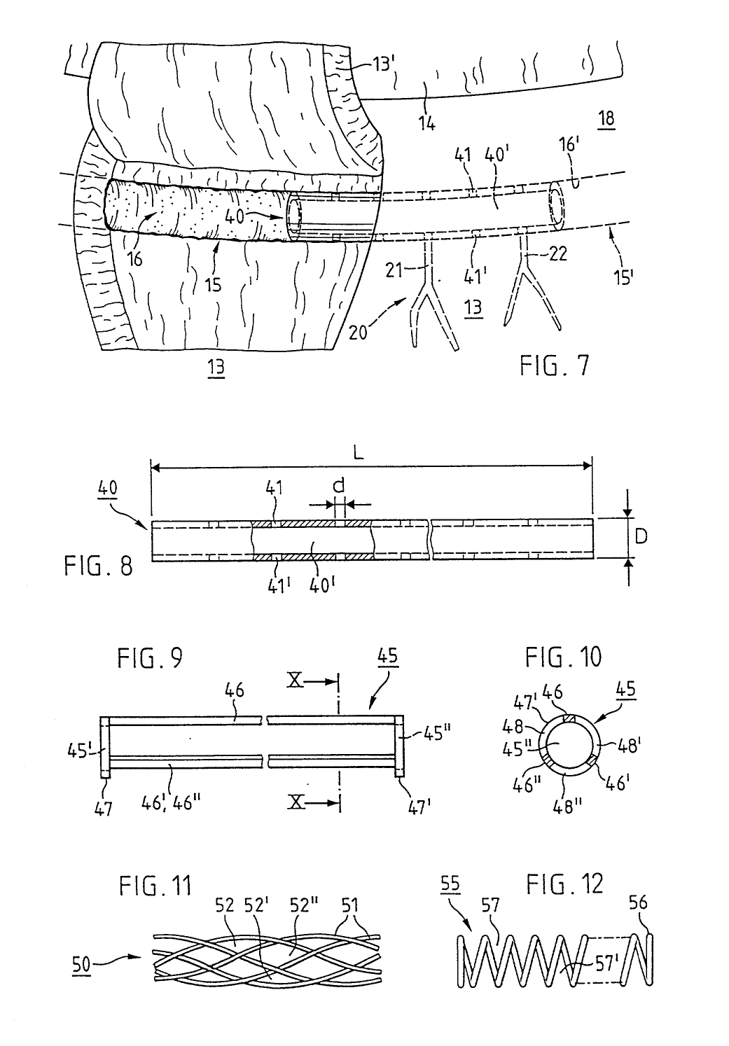

FIG. 1 shows an enlarged schematic vertical section through the anterior part of the eye 10. Evident in the anterior part of the eye are the cornea 11, the iris 12 with the sphincter muscle 12′ and 12″, the sclera 13, the lens 14 with the pupil 14′, and the zonular fibers 19 as well as Schlemm's canal 15 (sinus venosus sclerae) and the trabecular meshwork 18 (trabeculum comeosclerale) upstream thereto.

In a healthy eye, the drainage of the aqueous humor, which is constantly renewed and circulates according to arrows 1,1′ and 2,2′ from the posterior chamber H to the anterior chamber V, occurs in the iridocomeal angle V′ (angulus iridocomealis) following arrow direction 3 through the trabecular mesh-work 18 into the Schlemm's canal 15 and from there through the collector channels 21′,22′ (FIG. 4) or 21,22 (FIG. 7) of the subsequent natural outflow pathways 20,20′ (FIGS. 2,4,7) into the vein system (not shown). The resistance of the drainage system regulates the flow of the aqueous humor so that the pressure in the eye remains in a specific range tolerated by the tissue of the eye.

Under pathological conditions, the resistance may increase, whereby one of the factors is in Schlemm's canal. Schlemm's canal 15 may close such that the drainage of the aqueous humor is inhibited or completely blocked. As a result of the elevated resistance, pressure increases inside the eye so that the blood supply and consequently the function of the optic nerve is restricted. This disorder, generally known as “glaucoma”, often leads to blindness in one or both eyes.

FIG. 2 depicts the eye 10 schematically and shows the lens 14 with the pupil 14′, the partially depicted sclera 13, and the partially depicted Schlemm's canal 15 and a portion of the natural collector channel system 20,20′ connected therewith (aqueous humor canal system). For the surgical intervention, in a first phase, as depicted schematically in FIG. 2, the sclera 13 is microsurgically incised and a lamellar flap 13′ is formed which is folded upward for partial exposure of Schlemm's canal 15. During the course of the surgery, the flap portion 13′ of the sclera is held in this position by means, such as clamps or the like, which are not depicted.

In a second phase, as depicted schematically in FIG. 3, a probe 33, tubular in design and disposed on a schematically depicted connector 32, is introduced into the lumen 16 of Schlemm's canal 15. The connector 32 is linked with a schematically depicted injection apparatus 30 through a supply line (not shown). A hydrophilic material, for example a hydrophilic liquid 29, is injected into Schlemm's canal 15 by means of the injection apparatus 30 following the arrow direction 31 through the tubular probe 33 provided on the distal end with one or more outflow openings 33′. With the injected hydrophilic liquid 29, the virtually closed portion 15′ of Schlemm's canal 15 depicted schematically in FIG. 3 is hydraulically expanded.

As an improvement or supplement to the method, the portion 15″ of Schlemm's canal 15 opposite the portion 15′ already treated may (in a manner not shown in detail) be treated analogously and expanded using a probe designed preferably as a mirror image and introduced into the Schlemm's canal 15. FIG. 3 also depicts the trabecular meshwork 18 (trabecula) upstream from Schlemm's canal 15 with the schematically depicted trabecular mesh 18′.

At the time of the above described expansion of Schlemm's canal 15, openings (not depicted) created in its wall are simultaneously coated with the hydrophilic material injected, for example, such that the hydrophilic liquid 29 clinging to the walls of these openings, in the form of a film, prevents local contact between the rims of these openings which hinders the discharge of the aqueous humor.

At this point, reference is made to the fact that instead of the hydrophilic material or liquid, an appropriate biocompatible gaseous medium or even a mixture of the hydrophilic liquid and the gaseous medium may be used to expand Schlemm's canal.

As depicted schematically in FIG. 4, an implant supporting the inner wall 16′ connected to the hydraulic or pneumatic expansion and to optimize permanent passability and circulation of the aqueous humor is used in one portion 15″ of Schlemm's canal 15. As the first exemplary embodiment of an implant, a support element 35 with a long tube 36, which is inserted with its distal end 35″ into Schlemm's canal 15, is provided. On the other, proximal end 35′, the support element 35 is provided in the exemplary embodiment depicted with a collar 37 which fits closely against the inward face 13″ of scleral incision, whereby any displacement of the implanted support element 35 in Schlemm's canal 15 is prevented. The tube 36 is further provided with a number of throughholes 38, 38′ distributed axially and circumferentially spaced. The support element 35 is, as depicted schematically in FIG. 4, preferably positioned and implanted in the portion 15″ of Schlemm's canal 15 such that at least one of the throughholes 38,38′ connects with the small collector channels 21′, 22′ of the natural outflow pathways 20′.

The tube 36 of the support element 35 is shown in perspective in FIG. 5, and in a front elevational view and in partial cross-section in FIG. 6. The distributed throughholes 38,38′ are connected with the interior 36′ and the essentially torus-shaped collar 37. In the exemplary embodiment depicted, the circular collar 37 is, for example, placed on the tube 36 with a torus-shaped transition 37′, formed with a bell-shape or flair on it, for example, by suitable a suitable technique, such as utilizing a mandrel (not shown). In another exemplary embodiment, for improved insertion of the support element 35 into Schlemm's canal 15, the tube 36 can be tapered axially starting from the collar 37 or from the transition 37′ to the distal end 35″.

FIG. 7 shows the other portion 15′ of Schlemm's canal 15 with the support element 40 designed and implanted as a second embodiment of the present invention. The support element 40 is preferably positioned and implanted such that at least one of the outflow openings 41,41′, as depicted schematically in FIG. 7, is connected with the collector or small channels 21,22 of the natural outflow pathways 20. The aqueous humor penetrating through the trabecular meshwork 18 exits through Schlemm's canal 15 or through the interior 40′ of the support element 40 and through the openings 41′ and collector channels 21,22 of the subsequent natural outflow pathways 20.

FIG. 8 shows in partial cross-section the second exemplary embodiment of the tubular support element 40. The support element 40 is provided with multiple outflow openings 41,41′ spaced at intervals axially and arbitrarily distributed circumferentially or placed diametrically opposite each other and connected with the interior 40′.

FIGS. 9 and 10 depict a third embodiment of the support element 45 which has two end portions 47,47′, each provided with one opening 45′,45″ and designed as axially spaced toruses, between which are placed at least two, but preferably three webs 46, 46′ and 46″ placed circumferentially at intervals and linking the end portions 47,47′ to each other. In this embodiment, the recesses 48, 48′ and 48″ provided between the webs 46, 46′ and 46″ serve in each case as outflow openings for the aqueous humor to be drained substantially through the openings 45′ and 45″.

FIG. 11 depicts, as a fourth embodiment, the support element 50, which is designed essentially as a helicoidal network made of threads 51 which are interlinked, and are preferably formed of a stiff material. The network may, for example, be produced from relatively stiff plastic or metal threads or filaments 51 or from a biological material. The individual threads 51 of the network may also be interconnectedly wound counter to each other and in a helical shape. In this embodiment, the gaps 52,52′ and 52″ provided between the individual threads 51 serve respectively as outflow openings for the aqueous humor. The support element 50 may be designed such that it may be compressed for implantation and automatically expanded in the lumen 16 of Schlemm's canal 15 after implantation.

The interconnected metal threads or filaments 51 of the support element 50 (FIG. 11) are preferably made of a nickel-titanium alloy. These filaments 51 have a so-called shape memory effect, as a result of which the support element 50 designed as a network can be plastically deformed and with appropriate heating can be automatically returned to its original shape. The support element 50 with the thermal shape memory has the advantage that it can, for example, be inserted plastically deformed with a relatively small external diameter into the exposed Schlemm's canal at normal human body temperature and then, because of the normal body temperature is returned to its original form or shape.

FIG. 12 depicts, as another exemplary embodiment, the support element 55, which is, for example, produced from a single wire 56 wound in a helical shape produced from relatively stiff plastic or metal threads 56 or made of a noble metal, for example, a silver, gold, or platinum wire. In this embodiment, gaps 57 and 57′ provided between the individual turns serve respectively as outflow openings for the aqueous humor.

The support elements 35;40;45;50 or 55 designed, for example, as tubes or spirals made of suitable biocompatible material enable, in particular, due to their inherent flexibility, optimal adaptation to the natural shape of Schlemm's canal 15. The hollow cylindrical support elements 35;40;45;50 or 55 may, however, be coated with a suitable material, whereby desired biological reactions are generated or adverse biological reactions are reduced or completely prevented with the coating material.

In another embodiment, the support element 35;40;45;50 or 55 can be designed longitudinally somewhat arcuate. Also, as previously discussed, the support element 35;40;45;50;55 can be designed conically tapering longitudinally from one end to the other.

To illustrate the dimensions of the individual support elements 35;40;45;50 or 55 and the difficult manipulation thereof during implantation in the expanded lumen 16 of Schlemm's canal 15 (FIGS. 4, 7), reference is made at this point to the fact that the support elements have, for example, a length L of about 2.0 mm and an external diameter D of about 0.2 mm. The outflow openings 38, 38′ or 41, 41′ placed axially and at intervals circumferentially according to FIG. 5, 6 and 8 have an inner open diameter d of about 0.18 mm. The support elements 35;40;45;50 and 55 are, however, not restricted to the dimensions indicated above as examples.

Exemplary embodiments for introduction of the respective support elements include the following variations:

Variant I: After expansion, the injection apparatus 30 with the probe 33 is withdrawn from Schlemm's canal 15, and then the support element 35;40;45;50 or 55 is manually inserted into the lumen 16 of Schlemm's canal 15 by an appropriate technique, such as utilizing medical forceps or tweezers, or another surgical instrument and positioned (FIGS. 4, 7);

Variant II: The support element 35;40;45;50 or 55 is placed by means of a separable connection on the distal end of the probe 33 of the injection apparatus 30, and after the expansion of Schlemm's canal 15, is separated for the implantation by means not depicted;

Variant III: The distal end of the probe 33 of the injection apparatus 30 is designed as a separable support element 35; 40;45;50 or 55;

Variant IV: The substantially hollow cylindrical support element 35;40;45;50 or 55 is pushed onto the distal end of the probe 33 such that after the expansion of Schlemm's canal 15, the support element 35;40;45;50 or 55 is pushed by suitable means in a axial direction relative to the probe 33 into the lumen 16 of Schlemm's canal 15 and positioned.

Additional advantageous variants within the framework of the invention are also possible for implantation of the support element 35;40;45;50 or 55 in the lumen 16 of Schlemm's canal 15.

The invention is not restricted to the above described exemplary embodiments of the individual support elements 35;40; 45;50 or 55. Other advantageous designs of the support elements are also possible without going outside the basic idea of the invention. The support elements described in detail above and depicted are frequently also called endoprostheses. The combination of the expansion (hydraulic or pneumatic) and the opening of the essentially closed Schlemm's canal 15 depicted schematically in FIG. 3 with the subsequent implantation of the appropriately designed support element 35;40;45;50 or 55, in particular of the flexibly designed support element, is considered particularly advantageous.

With the support element 35 or 40, the lumen 16 of Schlemm's canal 15 is permanently held open, whereby the support element 35 or 40 is, for example, positioned such that at least one of the outflow openings 38, 38′ or 41,41′, as depicted schematically in FIG. 4 and FIG. 7, is connected with the collector channels 21′,22′ or 21,22 of the subsequent natural outflow pathways 20′ or 20. The aqueous humor penetrating into the trabecular meshwork 18 exits via Schlemm's canal 15 or via the lumen 36′ or 40′ of the support element 35 or 40 and via the openings 38′ or 41′ and collector channels 21′,22′ or 21,22 of the subsequent natural outflow pathways 20′ or 20.

Reference is made to the fact that in the lumen 16 of Schlemm's canal 15 at least one axially oriented support element 35;40;45;50;55 supportingly contacting the inner wall 16′ of Schlemm's canal 15 is implanted. If need be, there is also the possibility that two or more support elements are implanted in the deformed or obstructed Schlemm's canal 15. Here, it is advantageous if the respective implanted support element ensures a connection of Schlemm's canal 15 with at least one collector channel 21,22 or 21′,22′ of the subsequent natural outflow pathways 20 or 20′.

It is noted that the foregoing examples have been provided merely for the purpose of explanation and are in no way to be construed as limiting of the present invention. While the present invention has been described with reference to a preferred embodiment, it is understood that the words which have been used herein are words of description and illustration, rather than words of limitation. Changes may be made, within the purview of the appended claims, as presently stated and as amended, without departing from the scope and spirit of the present invention in its aspects. Although the present invention has been described herein with reference to particular means, materials and embodiment, the present invention is not in tended to be limited to the particulars disclosed herein, rather, the present invention extends to all functionally equivalent structures, methods and uses, such as are within the scope of the appended claims.

What is claimed is:

1. A device for holding an expanded lumen of Schlemm's canal in a permanently expanded position after Schlemm's canal is exposed in at least one place and expanded by a medium injected by an injection apparatus, comprising:
a support element comprising an elongated member that is axially and permanently orientable in Schlemm's canal, said elongated member comprising an interior and a plurality of outflow openings circumferentially and longitudinally distributed at intervals and connecting with said interior, said elongated member comprising a hollow cylindrical element, wherein said hollow cylindrical element is flexible for automatic adaptation to the lumen of Schlemm's canal in a direction of a theoretical longitudinal axis, and said elongated member being constructed and arranged to permit aqueous humor to permanently drain from Schlemm's canal through natural outflow pathways of the eye.

2. The device according to claim 1, wherein the elongated member comprises a long tube having a proximal end and a distal end, and said proximal end includes a contact collar flaring conically outward.

3. The device according to claim 1, wherein said elongated member has an arcuate shape similar to that of Schlemm's canal.

4. The device according to claim 1, wherein said elongated member is automatically deformable into an arcuate shape similar to that of Schlemm's canal.

5. The device according to claim 1, wherein said elongated member longitudinally tapers from said proximal end to said distal end.

6. The device according to claim 1, wherein said elongated member comprises two axially spaced torus-shaped end portions, each of said end portions including an outflow opening, and at least two webs circumferentially disposed at intervals between said end portions.

7. The device according to claim 6, wherein three such webs are circumferentially disposed at intervals between said end portions.

8. The device according to claim 1, wherein said elongated member comprises interlinked threads constructed and arranged as a hollow cylindrical network including outflow openings.

9. The device according to claim 8, wherein said network is plastically deformable, and is returnable to an original shape by heating.

10. The device according to claim 8, wherein said threads are composed of nickel-titanium alloy with thermal shape memory.

11. The device according to claim 8 wherein said threads are composed of a plastic heaving thermal shape memory.

12. The device according to claim 8, wherein said threads are individually wound with respect to each other forming said hollow cylindrical network.

13. The device according to claim 8, wherein said threads are bound to each other in a helicoidal network.

14. The device according to claim 8, wherein said hollow cylindrical network is capable of automatically expanding.

15. The device according to claim 1, wherein said elongated member comprises interlinked metal wires wound together as a hollow cylindrical network including outflow openings.

16. The device according to claim 1, wherein said elongated member is composed of a coil spring of a single wire wound in a helix.

17. The device according to claim 1, wherein said hollow cylindrical element is composed of a biocompatible material.

18. The device according to claim 17, wherein said biocompatible material comprises a member selected from the group consisting of plastic, stainless steel, noble metal and biological material.

19. The device according to claim 18, wherein said biocompatible material comprises a noble metal selected from the group consisting of silver, gold and platinum.

20. The device according to claim 1, wherein said hollow cylindrical element is coated with material capable of generating desired chemical reaction.

21. The device according to claim 1, wherein said hollow cylindrical element is coated with material capable of preventing an adverse biological reaction.

22. The device according to claim 1, wherein said hollow cylindrical element is connected to the injection apparatus via a separable connection.

23. The device according to claim 1, wherein said support element comprises a separable probe of the injection apparatus.

24. The device according to claim 1, wherein said support element comprises a hollow cylindrical support element capable of being pushed onto a probe of the injection apparatus, and can be implanted by axial movement of the probe relative to the support element for stable positioning into the expanded portion of Schlemm's canal.

25. A device for maintaining drainage of aqueous humor via the Schlemm's canal to the natural outflow pathways, comprising a support element sized and configured for permanent placement into the lumen of the Schlemm's canal, wherein the support element includes an elongated tubular member which has at least one opening in communication with the trabecular meshwork of the eye, and at least one opening in communication with the natural outflow pathways, when the support element is implanted in the lumen of the Schlemm's canal, so as to allow flow of aqueous humor from the trabecular meshwork to the lumen of the Schlemm's canal and from the lumen of the Schlemm's canal to the natural outflow pathways, wherein the tubular member is made of a material sufficiently flexible to allow the tubular member to automatically conform to a configuration of the lumen of the Schlemm's canal.

26. The device of claim 25, wherein the tubular member has one end formed with a contact collar flaring conically outward.

27. The device of claim 26, wherein the tubular member has another end and longitudinally tapers from the one end to the other end.

28. The device of claim 25, wherein the tubular member includes two axially spaced torus-shaped end portions, each of the end portions including an outflow opening, and at least two webs circumferentially disposed at intervals between the end portions.

29. The device of claim 28, wherein three such webs extend between the end portions.

30. The device of claim 25, wherein the tubular member includes interlinked threads constructed and arranged as a hollow cylindrical network.

31. The device of claim 30, wherein the network is plastically deformable, and returns to an original shape when exposed to heat.

32. The device of claim 30, wherein the threads are composed of nickel-titanium alloy with thermal shape memory.

33. The device of claim 30, wherein the threads are composed of a plastic having thermal shape memory.

34. The device of claim 30, wherein the threads are individually wound with respect to each other forming the hollow cylindrical network.

35. The device of claim 30, wherein the threads are bound to each other in a helicoidal network.

36. The device of claim 25, wherein the elongated tubular member includes interlinked metal wires wound together as a hollow cylindrical network.

37. The device of claim 25, wherein the tubular member is a coil spring made of a single wire wound to a helix.

38. The device of claim 25, wherein the support element is made of a biocompatible material.

39. The device of claim 38, wherein the biocompatible material is a material selected from the group consisting of plastic, stainless steel, noble metal and biological material.

40. The device of claim 39, wherein the noble metal is a metal s elected from the group consisting of silver, gold and platinum.
